(19) United States
(12) Patent Application Publication
(10) Pub. No.: US 2006/0167035 A1
(43) Pub. Date: Jul. 27, 2006

(54) Quinoline derivative, its use, production and pharmaceutical agents containing the latter

(76) Inventors: Wolfgang Schwede, Glienicke (DE); Stefan Jaroch, Berlin (DE); Benjamin Bader, Berlin (DE); Roman Hillig, Hamburg (DE); Antonius Ter Laak, Berlin (DE); Dieter Zopf, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/312,635

(22) Filed: Dec. 21, 2005

Related U.S. Application Data
(60) Provisional application No. 60/641,733, filed on Jan. 7, 2005.

(30) Foreign Application Priority Data
Dec. 22, 2004 (DE) 102004063223.5

(51) Int. Cl. A61K 31/4745; C07D 498/02; C07D 471/02

(52) U.S. Cl. 514/291; 546/81; 514/292

(57) ABSTRACT

This invention relates to a quinoline derivative with general formula A 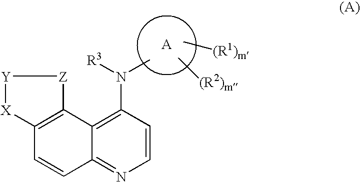
in which R¹, R², R³, X, Y, Z and A are indicated in the description and the claims, the use of the compounds of general formula A for treating various diseases as well as the production of compounds of general formula A.

This application claims the benefit of the filing date of U.S. Provisional Application Ser. No. 60/641,733 filed Jan. 7, 2005 which is incorporated by reference herein.

### DESCRIPTION

The invention relates to certain quinoline derivatives, their production and use as inhibitors of protein kinases, in particular Eph (erythropoetin-producing hepatoma amplified sequence) receptors for treating various diseases.

Protein tyrosine kinases catalyze the phosphorylation of specific tyrosine radicals in various proteins. Such phosphorylation reactions play a role in a number of cellular processes that are involved in the regulation of the growth and the differentiation of the cells involved. Protein tyrosine kinases are divided into receptor and non-receptor tyrosine kinases. The family of the receptor tyrosine kinases (RTKs) consists of 58 kinases (Manning, G. et al. 2002, Science 298, 1912-1934). RTKs have an extracellular ligand-binding domain, a transmembrane domain and an intracellular domain that generally contain the tyrosine kinase activity. RTKs mediate the signal relay of extracellular stimulators, such as, e.g., growth factors. The ligand bond leads to dimerization of the RTKs and the mutual auto-phosphorylation of their intracellular domains. Based on the cell type, specific intracellular binding proteins are thus recruited (i.a., non-receptor tyrosine kinases), via which a signal processing is carried out in the cell (Schlessinger, J. 2000, Cell 103, 211-225). The latter include receptor families of growth facors such as EGF (epidermal growth factor), VEGF (vascular endothelial growth factor), FGF (fibroblast growth factor), PDGF (platelet-derived growth factor) and NGF (nerve growth factor), as well as the insulin receptors and the large family of ephrin receptors, etc.

The ephrin (Eph) receptors make up the largest family within the RTKs. They are divided according to their sequential affinity and their ligand specificity into the group of EphA receptors (9 members) and the EphB receptors (6 members) (Kullander, K. and Klein, R. 2002, Nat. Rev. Mol. Cell Biol. 3, 475-486; Cheng, N. et al. 2002, Cyt. and Growth Factor Rev. 13, 75-85). Eph receptors are activated by membrane-fixed ligands of the EphrinA or EphrinB family. EphrinAs are anchored via glycolipids (GPI) in the cell membrane, while EphrinBs have a transmembrane region and an intracellular domain. The interaction between ephrins and the Eph receptors results in a bi-directional signal transfer in the ephrin-expressing cells and in the cells that carry the Eph receptor. Ephrins and Eph receptors play a role in a number of morphogenetic processes in embryonic development and in the adult organism. They are involved in embryonic pattern formation, in the development of the blood vessel system (Gerety, S. S. et al., 1999, Mol. Cell 4, 403-414) and in creating neuronal circuits (Flanagan, J. G. and Vanderhaeghen, P., 1998, Annu. Rev. Neurosci. 21, 306-354). In the adult organism, they are involved in the neovascularization process, e.g., in tumor development and in endometriosis, as well as in the morphogenesis of the intestinal epithelium (Battle, E. et al. 2002, Cell 111:251-63). On the cellular plane, they mediate migration, adhesion and juxtracrine cell contacts. Elevated expression of Eph receptors, such as, e.g., EphB2 and EphB4, was also observed in various tumor tissues, such as, e.g., breast tumors and tumors of the intestine (Nakamoto, M. and Bergemann, A. D. 2002, Mic. Res. Tech. 59, 58-67). Knock-out mice of EphB2, EphB3 and EphB4 show defects in the formation of the blood vessel system. The embryonic mortality of the EphB4-I-mice in embryonic stage d14 shows the special role of EphB4 in this process (Gerety, S. S.: et al. 1999, Mol. Cell 4, 403-414). A modulation of these receptors, e.g., by the inhibition of their kinase activity, results, for example, in that the tumor growth and/or the tumor metastasizing is suppressed either by a direct antitumoral action or by an indirect antiangiogenic action.

Non-receptor tyrosine kinases are present intracellularly in soluble form and are involved in the processing of extracellular signals (e.g., of growth factors, cytokines, antibodies, adhesion molecules) within the cell. They include, i.a., the families of Src (sarcoma) kinases, the Tec (tyrosine kinase expressed in hepatocellular-carcinoma) kinases, the Abl (Abelson) kinases and the Brk (breast-tumor kinase) kinases, as well as the focal adhesion kinase (FAK).

A modified activity of these protein tyrosine kinases can result in the most varied physiological disorders in the human organism and thus cause, e.g., inflammatory, neurological and oncological diseases.

In WO 01/19828 A, the most varied kinase inhibitors are disclosed.

In US 2004116388 A, triazine compounds that inhibit receptor tyrosine kinases are disclosed.

In WO 03/089434-A, imidazo[1,2a]pyrazin-8-yl-amines are disclosed, and in WO 04/00820 A, various aromatic monocyclic compounds that inhibit receptor tyrosine kinases are disclosed.

DE 24 27 409 A1 describes 9-(substituted amino)imidazo[4,5f]-quinoline as an active anthelmintic agent.

In EP 0 187 705 A2, imidazo[4,5f]-quinolines that have an immunomodulating action in infectious diseases are described. In U.S. Pat. No. 4,716,168, imidazo[4,5f]-quinolines with immunomodulating actions are also described. U.S. Pat. No. 5,506,235 A also discloses imidazo[4,5f]-quinolines with immunostimulating action.

Ferlin, M. G. et al. 2000, Biorganic & Med. Chem 8(6), 1415-1422 discloses pyrroloquinolines with cell growth-inhibiting properties.

In WO 04/006846 A, various quinazoline derivatives that inhibit receptor tyrosine kinases are disclosed.

Under receptor tyrosine kinase inhibitors, however, no Eph-receptor inhibitors are described.

The object of this invention is to provide compounds that inhibit receptor tyrosine kinases, in particular Eph receptors.

The object is achieved by quinoline derivatives with general formula A according to claim 1, the uses of the quinoline derivative according to claims 11 to 15, a process for the production of the quinoline derivative according to claim 16 as well as a pharmaceutical agent that contains the quinoline derivative according to claim 17. Advantageous embodiments are indicated in the subclaims.

This invention relates to a quinoline derivative with general formula A Quinoline derivative with general formula A: 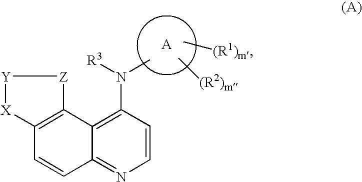
whereby
A is selected from the group that comprises —C₆-C₁₂-aryl, —C₅-C₁₈-heteroaryl, —C₃-C₁₂cycloalkyl and —C₃-C₁₂-heterocycloalkyl,
R¹ and R² are the same or different and are selected in one or more places, independently of one another, from the group that comprises hydrogen, hydroxy, halogen, nitro, cyano, —C₁-C₆-alkyl, —C₁-C₄-hydroxyalkyl, —C₂-C₆-alkenyl, —C₂-C₆-alkinyl, —C₃-C₁₀-cycloalkyl, —C₃-C₁₂-heterocycloalkyl, —C₆-C₁₂-aryl, —C₅-C₁₈-heteroaryl, —C₁-C₆-alkoxy, —C₁-C₆-alkoxy-C₁-C₆-alkoxy, —C₁-C₆-alkoxy-C₁-C₆-alkyl, —C₁-C₆-alkoxy-C₁-C₆-alkoxy-C₁-C₆-alkyl, —(CH₂)ₙ—C₆-C₁₂-aryl, —(CH₂)ₙ—C₅-C₁₈-heteroaryl, —(CH₂)ₙ—C₃-C₁₀-cycloalkyl, —(CH₂)ₙ—C₃-C₁₂-heterocycloalkyl, -phenylene-(CH₂)ₚ—R⁶, —(CH₂)ₚPO₃(R⁶)₂, —(CH₂)ₚ—NR⁵R⁶, —(CH₂)ₚ—NR⁴COR⁵, —(CH₂)ₚ—NR⁴CSR⁵, —(CH₂)ₚ—NR⁴S(O)R⁵, —(CH₂)ₚ—NR⁴S(O)₂R⁵, —(CH₂)ₚ—NR⁴CONR⁵R⁶, —(CH₂)ₚ—NR⁴COOR⁵, —(CH₂)ₚ—NR⁴C(NH)NR⁵R⁶, —(CH₂)ₚ—NR⁴CSNR⁵R⁶, —(CH₂)ₚ—NR⁴S(O)NR⁵R⁶, —(CH₂)ₚ—NR⁴S(O)₂NR⁵R⁶, —(CH₂)ₚ—COR⁵, —(CH₂)ₚ—CSR⁵, —(CH₂)ₚ—S(O)R⁵, —(CH₂)ₚ—S(O)(NH)R⁵, —(CH₂)ₚ-S(O)₂R⁵, —(CH₂)ₚ—S(O)₂NR⁵R⁶, —(CH₂)ₚ—SO₂OR⁵, —(CH₂)ₚ—CO₂R⁵, —CH₂)ₚ—CONR⁵R⁶, —(CH₂)ₚ—CSNR⁵R⁶, —OR⁵, —(CH₂)ₚ—SR⁵ and —CR⁵(OH)—R⁶, whereby —C₁-C₆-alkyl, —C₂-C₆-alkenyl, —C₂-C₆-alkinyl, —C₃-C₁₀-cycloalkyl, —C₃-C₁₂-heterocycloalkyl, —C₆-C₁₂-aryl, —C₅-C₁₈-heteroaryl and/or —C₁-C₆-alkoxy are unsubstituted or are substituted in one or more places, independently of one another, with hydroxy, halogen, nitro, cyano, phenyl, —NR⁵R⁶, alkyl and/or —OR⁵, whereby the carbon skeleton of the —C₃-C₁₀-cycloalkyl and the —C₁-C₁₀-alkyl can contain nitrogen, oxygen or sulfur atoms and/or C═O groups and/or one or more double bonds in one or more places, independently of one another, and/or R¹ and R² optionally form a bridge with one another that consists of 3-10 methylene units, whereby up to two methylene units are optionally replaced by O, S and/or —NR⁴,
X, Y, Z are the same or different and are selected independently of one another from the group that comprises —CR³═, —CR³R⁴—, —C(O)—, —N═, —S—, —O—, —NR³—, —S(O)₂—, —S(O)— and —S(O)NH)— and single or double bonds are found between X, Y and Z,
R³ is hydrogen, —C₁-C₁₀-alkyl or —C₁C₁₀-alkanoyl,
R⁴ is hydrogen or —C₁-C₁₀-alkyl,
R⁵ and R⁶ are the same or different and are selected, independently of one another, from the group that comprises hydrogen, —C₁-C₁₀-alkyl, —C₂-C₁₀-alkenyl, —C₂-C₁₀-alkinyl, —C₁-C₆-alkoxy, —C₃-C₁₀-cycloalkyl, —C₃-C₁₂-heterocycloalkyl, —C₆-C₁₂-aryl and —C₅-C₁₈-heteroaryl, whereby —C₁-C₁₀-alkyl, —C₂-C₁₀-alkenyl, —C₂-C₁₀-alkinyl, —C₁-C₆-alkoxy, —C₃-C₁₀-cycloalkyl, —C₃-C₁₂-heterocycloalkyl, —C₆-C₁₂-aryl and/or —C₅-C₁₈-heteroaryl are unsubstituted or [are substituted] in one or more places, independently of one another, with hydroxy, halogen, cyano, nitro, —OR⁷, —NR⁷R⁸, —C(O)NR⁷R⁸, —C(O)OR⁷ and/or —C₁-C₆-alkyl, whereby —C₁-C₆-alkyl is unsubstituted or [is substituted] in one or more places, independently of one another, with halogen, hydroxy, cyano, —NR⁷R⁸, —OR⁷ and/or phenyl; and/or R⁵ and R⁶ optionally form a bridge with one another that consists of 3-10 methylene units, whereby up to two methylene units optionally are replaced with O, S and/or NR⁴,
R⁷ and R⁸ are the same or different and are selected, independently of one another, from the group that comprises hydrogen, —C₁-C₄-alkyl, —C₆-C₁₂-aryl and —C₅-C₁₈-heteroaryl, whereby alkyl, aryl, or heteroaryl is unsubstituted or [is substituted] in one or more places, independently of one another, with halogen and/or alkoxy, or R⁷ and R⁸ optionally form a bridge with one another that consists of 3-10 methylene units, whereby up to two methylene units optionally are replaced with O, S and/or —NR⁴;
m′, m″=0-4, independently of one another,
n=1-6,
p=0-6, as well as
   their N-oxides, solvates, hydrates, stereoisomers, diastereomers, enantiomers and salts.

If X, Y, and Z, independently of one another, mean one, two or three N, the understanding holds true that
1. The skeleton in partial grouping X—Y—X is not N—CH—N, CH—N—N or N—N—N, and
2. X is not NH, if Y and Z are simultaneously CH in each case.

It was found that the compounds according to the invention can inhibit receptor tyrosine kinases, in particular Eph receptors.

The partial grouping CH—N—N and N—N—N that is mentioned in the last-mentioned section under 1. was described in the above-mentioned U.S. Pat. No. 5,506,235 A. Substances with this partial grouping have an immunostimulating action according to U.S. Pat. No. 5,506,235 A. The partial grouping N—CH—N that is mentioned in the last-mentioned section under 1. is described in DE 24 27 409 A1, EP 0 187 705 A2 or U.S. Pat. No. 4,716,168. Substances with this partial grouping have an anthelmintic action according to DE 24 27 409 A1 and an immunomodulating action according to EP 0 187 705 A2 and U.S. Pat. No. 4,716,168. Compounds with cell growth-inhibiting properties that fall under criteria cited in the last-mentioned section under 2. are described in Ferlin, M. G., et al. 2000, Bioorganic & Med. Chem. 8(6), 1415-1422.

In all documents described in this section, however, no Eph receptor inhibitors are disclosed.

Alkyl is defined in each case as a straight-chain or branched alkyl radical, such as, for example, methyl, ethyl, propyl, isopropyl, butyl, isobutyl, sec-butyl, tert-butyl, pentyl, isopentyl, hexyl, heptyl, octyl, nonyl and decyl.

Alkoxy is defined in each case as a straight-chain or branched alkoxy radical, such as, for example, methyloxy, ethyloxy, propyloxy, isopropyloxy, butyloxy, isobutyloxy, sec-butyloxy, pentyloxy, isopentyloxy, hexyloxy, heptyloxy, octyloxy, nonyloxy or decyloxy.

The alkenyl substituents are in each case straight-chain or branched, whereby, for example, the following radicals are meant: vinyl, propen-1-yl, propen-2-yl, but-1-en-1-yl, but-1-en-2-yl, but-2-en-1-yl, but-2-en-2-yl, 2-methyl-prop-2-en-1-yl, 2-methyl-prop-1-en-1-yl, but-1-en-3-yl, but-3-en-1-yl, or allyl.

Alkinyl is defined in each case as a straight-chain or branched alkinyl radical that contains two to six, preferably two to four C atoms. For example, the following radicals are suitable: ethinyl, propin-1-yl, propin-3-yl, but-1-in-1-yl, but-1-in-4-yl, but-2-in-1-yl, and but-1-in-3-yl.

As a cycloalkyl that can contain one or more heteroatoms such as sulfur, nitrogen or oxygen, the following, e.g., can be mentioned: oxiranyl, oxethanyl, aziridinyl, azetidinyl, tetrahydrofuranyl, pyrrolidinyl, dioxolanyl, imidazolidinyl, pyrazolidinyl, dioxanyl, piperidinyl, morpholinyl, dithianyl, thiomorpholinyl, piperazinyl, triethianyl, or quinuclidinyl.

Cycloalkyls are defined as monocyclic alkyl rings, such as cyclopropyl, cyclobutyl, cyclopentyl, cyclohexyl or cycloheptyl, but also bicyclic rings or tricyclic rings, such as, for example, adamantanyl. The cycloalkyl rings can be unsubstituted or substituted in one or more places.

Cycloalkyls according to this invention contain C₃-C₁₂ hydrocarbon atoms; cycloalkyls with C₃-C₁₀-hydrocarbon atoms are preferred, and cycloalkyls with C₃-C₆-hydrocarbon atoms are especially preferred.

An aryl radical in each case has 6-12 carbon atoms. The radical can be monocyclic or bicyclic, for example naphthyl, biphenyl and in particular phenyl.

The heteroaryl radical comprises an aromatic ring system, which in each case contains 5-18 ring atoms, preferably 5 to 10 ring atoms, and especially preferably 5 to 7 ring atoms, and instead of carbon contains one or more of the same or different heteroatoms from the group oxygen, nitrogen or sulfur. The radical can be monocyclic, bicyclic or tricyclic and in addition in each case can be benzocondensed. Only those combinations are meant, however, that are useful from the viewpoint of one skilled in the art, in particular in reference to ring strain.

The heteroaryl rings can be unsubstituted or substituted in one or more places. By way of example, there can be mentioned: thienyl, furanyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, triazinyl as well as benzo derivatives of these radicals, such as, e.g., 1,3-benzodioxolyl, benzofuranyl, benzothienyl, benzoxazolyl, benzimidazolyl, indazolyl, indolyl, isoindolyl, oxepinyl, azocinyl, indolizinyl, indolyl, isoindolyl, indazolyl, benzimidazolyl, purinyl, quinolinyl, isoquinolinyl, cinnolinyl, phthalazinyl, quinazolinyl, quinoxalinyl, naphthyridinyl, pteridinyl, carbazolyl, acridinyl, phenazinyl, phenothiazinyl, phenoxazinyl, xanthenyl, etc.

Halogen is defined in each case as fluorine, chlorine, bromine or iodine.

C₃-C₁₂-Heterocycloalkyl stands for an alkyl ring that comprises 3-12 carbon atoms, preferably 3 to 10 carbon atoms, and especially preferably 3 to 6 carbon atoms, which is interrupted by at least one of the following atoms nitrogen, oxygen and/or sulfur in the ring, and which optionally can be interrupted by one or more of the same or different —(CO)—, —SO— or —SO₂— groups in the ring and optionally contains one or more double bonds in the ring. Only those combinations are meant, however, that are useful from the standpoint of one skilled in the art, in particular with reference to the ring strain. According to this invention, C₃-C₁₂-heterocycloalkyls are monocyclic, but also bicyclic or tricyclic. As monocyclic heterocycles, e.g., there can be mentioned: oxiranyl, oxethanyl, aziridinyl, azetidinyl, tetrahydrofuranyl, pyrrolidinyl, dioxolanyl, imidazolidinyl, pyrazolidinyl, dioxanyl, piperidinyl, morpholinyl, dithianyl, thiomorpholinyl, piperazinyl, trithianyl, quinuclidinyl, etc.

Thus, as in this application, and for example in connection with the definition of “C₁-C₁₀-alkyl,” “C₁-C₁₀” refers to an alkyl group with an infinite number of 1 to 10 carbon atoms, .i.e., 1, 2, 3, 4, 5, 6, 7, 8, 9 or 10 carbon atoms. In addition, the definition “C₁-C₁₀” is interpreted such that any possible partial area, such as, for example, C₁-C₁₀, C₂-C₉, C₃-C₈, C₄-C₇, C₅-C₆, C₁-C₂, C₁-C₃, C₁-C₄, C₁-C₅, C₁-C₆, C₁-C₇, C₁-C₈, C₁-C₉, C₁-C₁₀ preferably C₁-C₂, C₁-C₃, C₁-C₄, C₁-C₅, C₁-C₆; preferably C₁-C₄ in the definition, is co-contained.

Analogously to this and for example in connection with the definition of “C₂-C₁₀-alkenyl” and “C₂-C₁₀-alkinyl,” “C₂-C₁₀” refers to an alkenyl group or an alkinyl group with an endless number of 2 to 10 carbon atoms, i.e., 2, 3, 4, 5, 6, 7, 8, 9, or 10 carbon atoms. The definition of “C₂-C₁₀” is interpreted such that any possible partial area, such as, for example, C₂-C₁₀, C₃-C₉, C₄-C₈, C₅-C₇, C₂-C₃, C₂-C₄, C₂-C₅, C₂-C₆, C₂-C₇, C₂-C₈, C₂-C₉, preferably C₂-C₄, is co-contained in the definition.

For example in connection with the definition of “C₁-C₆-alkoxy,” “C₁-C₆” further refers to an alkoxy group with an endless number of 1 to 6 carbon atoms, i.e., 1, 2, 3, 4, 5 or 6 carbon atoms. The definition “C₁-C₆” is interpreted such that any possible partial area, such as, for example C₁-C₆, C₂-C₅, C₃-C₄, C₁-C₂, C₁-C₃, C₁-C₄, C₁-C₅, C₁-C₆; preferably C₁-C₄, is co-contained in the definition.

All reference data of the application not explicitly cited here are defined analogously to the areas “C₁-C₁₀,” “C₂-C₁₀” and “C₁-C₆,” mentioned by way of example above.

Isomers are defined as chemical compounds of the same summation formula but of different chemical structure. In general, constitutional isomers and stereoisomers are distinguished. Constitutional isomers have the same summation formula, but are distinguished by the way in which their atoms or atom groups are linked. These include functional isomers, position isomers, tautomers or valence isomers. Stereoisomers basically have the same structure (constitution) and thus also the same summation formula, but are distinguished by the spatial arrangement of the atoms. In general, configuration isomers and conformation isomers are distinguished. Configuration isomers are stereoisomers that can be converted into one another only by bond breaking. These include enantiomers, diastereomers and E/Z (cis/trans) isomers. Enantiomers are stereoisomers that behave toward one another like image and mirror image and do not have any plane of symmetry. All stereoisomers that are not enantiomers are referred to as diastereomers. E/Z (cis/trans) isomers on double bonds are a special case. Conformation isomers are stereoisomers that can be converted into one another by the rotation of single bonds. To differentiate the types of isomerism from one another, see also the IUPAC Rules, Section E (Pure Appl. Chem. 45, 11-30, 1976).

The quinoline derivatives with general formula A according to the invention also contain the possible tautomeric forms and comprise the E or Z isomers, or, if a chiral center is present, also the racemates and enantiomers. Double-bond isomers are also defined among the latter.

The quinoline derivatives according to the invention can also be present in the form of solvates, in particular of hydrates, whereby the compounds according to the invention consequently contain polar solvents, in particular water, as structural elements of the crystal lattice of the compounds according to the invention. The portion of polar solvent, in particular water, can be present in a stoichiometric or else unstoichiometric ratio. In the case of stoichiometric solvates, hydrates, we also speak of hemi-(semi), mono-, sesqui-, di-, tri-, tetra-, penta-, etc., solvates or hydrates.

N-oxide means that at least one nitrogen of the compounds of general formula A according to the invention can be oxidized.

If an acid group is included, the physiologically compatible salts of organic and inorganic bases are suitable as salts, such as, for example, the readily soluble alkali and alkaline-earth salts, as well as salts of N-methyl-glucamine, di-methyl-glucamine, ethyl-glucamine, lysine, 1,6-hexadiamine, ethanolamine, glucosamine, sarcosine, serinol, tris-hydroxy-methyl-amino-methane, aminopropanediol, Sovak base, or 1-amino-2,3,4-butanetriol.

If a basic group is included, the physiologically compatible salts of organic and inorganic acids, such as hydrochloric acid, sulfuric acid, phosphoric acid, citric acid, tartaric acid, i.a., are suitable.

Functional groups can optionally be protected by protective groups during the reaction sequence. Such protective groups can be, i.a., esters, amides, ketals/acetals, nitro groups, carbamates, alkyl ethers, allyl ethers, benzyl ethers or silyl ethers. As components of silyl ethers, i.a., compounds, such as, e.g., trimethylsilyl (TMS), tert-butyl-dimethyl silyl (TBDMS), tert-butyl-diphenylsilyl (TBDPS), triethylsilyl (TES), etc., can occur. Their production is described in the experimental part.

Preferred are quinoline derivatives with the above-mentioned general formula A, provided that if X, Y, and Z, independently of one another, mean one, two or three N,
1. the skeleton in the partial grouping X—Y—Z is not N—N—CH, N—CH—N, CH—N—N or N—N—N, and
2. X is not NH, if Y and Z simultaneously are CH in each case.

Preferred are quinoline derivatives with the above-mentioned general formula A, in which:
R¹ and R² are the same or different and are selected in one or more places, independently of one another, from the group that comprises hydrogen, hydroxy, halogen, nitro, cyano, —C₁-C₆-alkyl, —C₁-C₄-hydroxyalkyl, —C₆-C₁₂-aryl, —C₁-C₆-alkoxy, —NR⁵R⁶, —NR⁴COR⁵, —NR⁴S(O)R⁵, —NR⁴S(O)₂R⁵, —NR⁴CONR⁵R⁶, —NR⁴S(O)NR⁵R⁶, —NR⁴S(O)₂NR⁵R⁶, —COR⁵, COOR⁵, —S(O)R⁵, —S(O)(NH)R⁵, —S(O)₂R⁵, —S(O)₂NR⁵R⁶, —CO₂R⁵, —CONR⁵R⁶, —OR⁵ and —CR⁵(OH)—R⁶, and
m′, m″=0-3, independently of one another.
R³ is preferably hydrogen in general formula A.
A in general formula A is preferably phenyl.

Compounds of general formula A in which the ring A is phenyl and R¹ and R² are the same or different and are selected in one or more places, independently of one another, from the group that comprises hydrogen, hydroxy, halogen, nitro, amino, cyano, —C₁-C₆-alkyl, —C₁-C₄-hydroxyalkyl, —C₁-C₆-alkoxy, —NH—C(O)—NH-aryl, —C₁-C₄-alkyl-CO—NH—, —COOR⁵ and preferably —COOR^{N}, whereby R^{N} stands for H, alkyl, alkenyl, cycloalkyl, or aryl, —CR⁵(OH)—R⁶ and —CONH₂, and
m′,m″=0-3, independently of one another,
   are especially preferred.

Quite especially preferred in this case are compounds in which R¹ and R² are the same or different and are selected in one or more places, independently of one another, from the group that comprises hydrogen, hydroxy, halogen, nitro, amino, cyano, —CH₃, —C₂H₅, CH₃O—, C₂H₅O—, HOCH₂—, CH₃CONH—, —NH—C(O)—NH-phenyl, —COOH and —CONH₂.

In addition, quinoline derivatives with general formula A, in which X, Y and Z, independently of one another, are selected from the group that comprises —CR⁴═, —CR⁴R⁵—, —C(O)—, —N═, —S—, —O—, —NR⁴—, —S(O)₂—, —S(O)— and —S(O)NH—, whereby N, S or O does not occur in several places in the ring, are preferred. In this case, the ring A is preferably phenyl and m′ and m″=0-2.

In addition, compounds of general formula A, in which X, Y and Z stand for —S(O)₂—, —S—, —NH—, —CH═, —C(CH₃)═ and/or —CH₂—, are preferred.

The skeleton in the partial grouping X—Y-Z in the quinoline derivative with general formula A is quite especially preferably selected from the group that comprises —S—CH═CH—, —S—C(C₁-C₆-alkyl)═N— and preferably —S—C(C₁-C₃-alkyl)═N—, and still more preferably —S—C(CH₃)═N—, —S(O)₂—CH₂—CH₂— and —CH═CH—S—.

Most preferred are the following compounds:
1) 4-Methyl-3-(thieno[3,2-f]quinolin-9-ylamino)-phenol
2) 4-Methyl-3-(2-methyl-thiazolo[4,5-f]quinolin-9-ylamino)-phenol
3) 4-Methyl-3-(thieno[2,3-f]quinolin-9-yl-amino)phenol
4) 3-(3,3-Dioxo-2,3-dihydro-1H-3λ⁶-thieno[3,2-f]quinolin-9-ylamino)-4-methyl-phenol
5) 3-(3,3-Dioxo-2,3-dihydro-1H-3λ⁶-thieno[3,2-f]quinolin-9-ylamino)-phenol
6) 4-(3,3-Dioxo-2,3-dihydro-1H-3λ⁶-thieno[3,2-f]quinolin-9-ylamino)-3-methyl-phenol
7) 2-(3,3-Dioxo-2,3-dihydro-1H-3λ⁶-thieno[3,2-f]quinolin-9-ylamino)-phenol
8) 4-(3,3-Dioxo-2,3-dihydro-1H-3λ⁶-thieno[3,2-f]quinolin-9-ylamino)-phenol
9) [3-(3,3-Dioxo-2,3-dihydro-1H-3λ⁶-thieno[3,2-f]quinolin-9-ylamino)phenyl]-methanol
10) 3-(3,3-Dioxo-2,3-dihydro-1H-3λ⁶-thieno[3,2-f]quinolin-9-ylamino)-benzoic acid
11) 3-(3,3-Dioxo-2,3-dihydro-1H-3λ⁶-thieno[3,2-f]quinolin-9-ylamino)-benzamide
12) (3,3-Dioxo-2,3-dihydro-1H-3λ⁶-thieno[3,2-f]quinolin-9-yl)-(3-methoxyphenyl)amine
13) N-[3-(3,3-Dioxo-2,3-dihydro-1H-3λ⁶-thieno[3,2-f]quinolin-9-ylamino)-phenyl]-acetamide
14) 3-(3,3-Dioxo-2,3-dihydro-1H-3λ⁶-thieno[3,2-f]quinolin-9-ylamino)-5-methoxyphenol
15) 5-(3,3-Dioxo-2,3-dihydro-1H-3λ⁶-thieno[3,2-f]quinolin-9-ylamino)-2-methylphenol
16) 3-(3,3-Dioxo-2,3-dihydro-1H-3λ⁶-thieno[3,2-f]quinolin-9-ylamino)-2-methylphenol
17) 3-(3,3-Dioxo-2,3-dihydro-1H-3λ⁶-thieno[3,2-f]quinolin-9-ylamino)-5-methyl-phenol
18) 4-Chloro3-(3,3-dioxo-2,3-dihydro-1H-3λ⁶-thieno[3,2-f]quinolin-9-ylamino)-5-methyl-phenol
19) 2-(3,3-Dioxo-2,3-dihydro-1H-3λ⁶-thieno[3,2-f]quinolin-9-ylamino)-4-methoxy-phenol
20) (3,3-Dioxo-2,3-dihydro-1H-3λ⁶-thieno[3,2-f]-quinolin-9-yl)-(2-methyl-5-nitrophenyl)-amine
21) [3-(3,3-Dioxo-2,3-dihydro-1H-3λ⁶-thieno[3,2-f]quinolin-9-ylamino)-4-methoxy-phenyl]-methanol
22) 1-[3-(3,3-Dioxo-2,3-dihydro-1H-3λ⁶-thieno[3,2-f]quinolin-9ylamino)-phenyl]-3-phenyl-urea
23) 1-[4-(3,3-Dioxo-2,3-dihydro-1H-3λ⁶-thieno[3,2-f]quinolin-9-ylamino)-phenyl]-3-phenyl-urea
24) (3,5-Dimethoxy-phenyl)-(3,3-dioxo-2,3-dihydro-1H-3λ⁶-thieno[3,2-f]quinolin-9-yl)-amine
25) (3,3-Dioxo-2,3-dihydro-1H-3λ⁶-thieno[3,2-f]-quinolin-9-yl)-(3,4,5-trimethoxy-phenyl)-amine
26) N³-(3,3-Dioxo-2,3-dihydro-1H-3λ⁶-thieno[3,2-f]quinolin-9-yl)-4-methyl-phenyl-1,3-diamine
27) 1-[3-(3,3-Dioxo-2,3-dihydro-1H-3λ⁶-thieno[3,2-f]quinolin-9-ylamino)-4-methyl-phenyl]-3-urea

The quinoline derivatives with general formula A according to the invention inhibit receptor tyrosine kinases, in particular Eph kinases, and this also accounts for their action, for example, in the treatment of diseases in which angiogenesis, lymphangiogenesis or vasculogenesis plays a role, in diseases of the blood vessels, diseases that are caused by a hyperproliferation of body cells or chronic or acute neurodegenerative diseases. These quinoline derivatives with general formula A can accordingly be used as pharmaceutical agents.

Treatments are preferably performed on humans, but also on related mammal species, such as, e.g., dogs and cats.

Angiogenic and/or vasculogenic diseases can be treated by the growth of the blood vessels being inhibited (antiangiogenic) or promoted (proangiogenic). Antiangiogenic uses are carried out, e.g., in the case of tumor angiogenesis, endometriosis, in diabetic-related or other retinopathies or in age-related macular degeneration. Proangiogenic uses are carried out in, e.g., myocardial infarction or acute neurodegenerative diseases by ischemias of the brain or neurotraumas.

Blood vessel diseases are defined as stenoses, arterioscleroses, restenoses or inflammatory diseases, such as rheumatic arthritis.

Hyperproliferative diseases are defined as solid tumors, non-solid tumors or non-carcinogenic cell hyperproliferation in the skin, whereby solid tumors are defined as, i.a., breast tumors, colon tumors, kidney tumors, lung tumors and/or brain tumors. Non-solid tumors are defined as, i.a., leukemias, and non-carcinogenic cell hyperproliferation in the skin is defined as, i.a., psoriasis, eczema, scleroderma or benign hypertrophy of the prostate.

Chronic neurodegenerative diseases are defined as, i.a., Huntington's disease, amyotrophic lateral sclerosis, Parkinson's disease, AIDS-induced dementia or Alzheimer's disease.

Use of the quinoline derivatives with general formula A can also be used for diagnostic purposes in vitro or in vivo for identifying receptors in tissues by means of autoradiography and/or PET.

In particular, the substances can also be radiolabeled for diagnostic purposes.

To use the quinoline derivatives according to the invention as pharmaceutical agents, the latter are brought into the form of a pharmaceutical preparation, which in addition to the active ingredient for enteral or parenteral application contains suitable pharmaceutical, organic or inorganic inert carrier materials, such as, for example, water, gelatin, gum Arabic, lactose, starch, magnesium stearate, talc, vegetable oils, polyalkylene glycols, etc. The pharmaceutical preparations can be present in solid form, for example as tablets, coated tablets, suppositories or capsules, or in liquid form, for example as solutions, suspensions or emulsions. They optionally contain, moreover, adjuvants, such as preservatives, stabilizers, wetting agents or emulsifiers; salts for changing the osmotic pressure, or buffers.

These pharmaceutical preparations are also subjects of this invention.

For parenteral application, in particular injection solutions or suspensions, in particular aqueous solutions of the active compounds in polyhydroxyethoxylated castor oil, are suitable.

As carrier systems, surface-active adjuvants, such as salts of bile acids or animal or plant phospholipids, but also mixtures thereof as well as liposomes or their components, can also be used.

For oral application, in particular tablets, coated tablets or capsules with talc and/or hydrocarbon vehicles or hydrocarbon binders, such as, for example, lactose, corn or potato starch, are suitable. The application can also be carried out in liquid form, such as, for example, as a juice, to which optionally a sweetener is added.

The enteral, parenteral and oral applications are also subjects of this invention.

The dosage of the active ingredients can vary depending on the method of administration, age and weight of the patient, type and severity of the disease to be treated and similar factors. The daily dose is 0.5-1,000 mg, whereby the dose can be given as an individual dose to be administered once or divided into two or more daily doses.

Pharmaceutical agents for treating the above-cited diseases that contain at least one quinoline derivative with general formula A, whereby the pharmaceutical agent optionally can contain suitable formulation substances and vehicles, are also subjects of this invention.

If the production of the starting compounds is not described, the latter are known to one skilled in the art or can be produced analogously to known compounds or processes that are described here. It is also possible to perform all reactions described here in parallel reactors or by means of combinatory operating procedures.

According to commonly used methods, such as, for example, crystallization, chromatography or salt formation, the isomer mixtures can be separated into enantiomers or E/Z isomers.

The production of salts is carried out in the usual way by a solution of the compound with general formula A being mixed with the equivalent amount of or an excess of a base or acid, which optionally is in solution, and the precipitate being separated or the solution being worked up in the usual way.

The process for the production of the quinoline derivatives according to the invention is also a subject of this invention.

The intermediate products that are preferably used for the production of the quinoline derivatives with general formula A according to the invention are the following compounds with general formulas I to VI.
Production of the Compounds According to the Invention 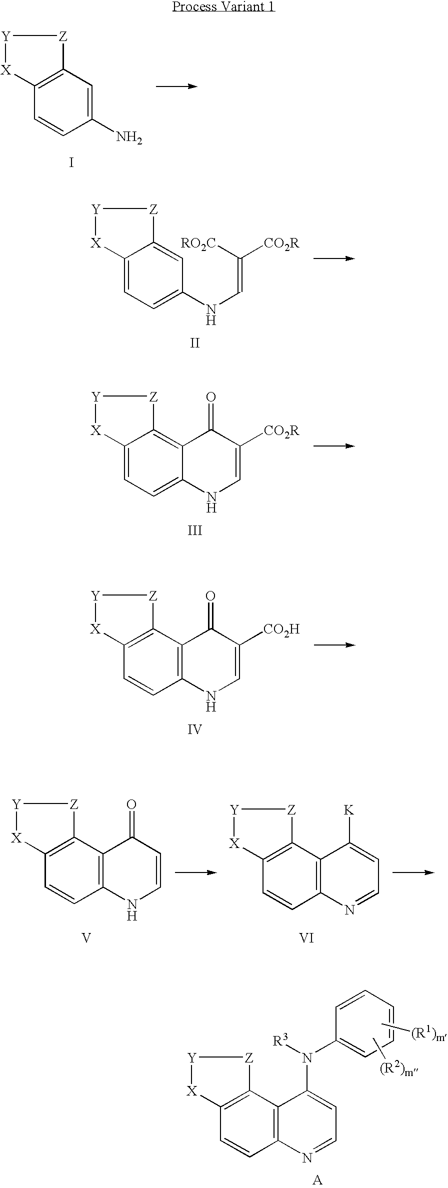
Diagram 1

Quinoline derivatives with general formula A according to the invention can be produced, for example, in the way shown in Diagram 1, in which radical K can be, for example, halogen or —OS(O)₂CₙF₂ₙ₊₁ with n=1-3, and radical R can be methyl or ethyl, and radicals X, Y and Z have the same meaning as in general formula A. The required starting materials are either commercially available or are produced according to processes that are known in the literature or analogously to processes that are known in the literature.

By addition of a compound with general formula I to a dialkylalkoxymethylene malonate, e.g., diethylethoxymethylene malonate, compounds with general formula II are formed. These compounds are then preferably cyclized under thermal conditions to compounds with general formula III. With these cyclizations, acids or Lewis acids can also be used. Then, the ester is saponified, whereby compounds with general formula IV are obtained that then are preferably decarboxylated under thermal conditions, whereby compounds with general formula V are produced. As an alternative, a direct decarboxylation of the alkyl esters with general formula III can also be performed. In addition to the mentioned thermal conditions, other processes for decarboxylation that are known in the literature both originating from compounds with general formula III as well as originating from compounds with general formula IV can also be used. Compounds with general formula VI are then produced by, e.g., reaction with thionyl chloride (for K═Cl) or perfluoroalkylsulfonic acid anhydrides (for K=perfluoroalkylsulfonyl). Compounds with general formula A can then be produced by addition of amines ((R¹)_{m′}, (R²)_{m″}ArNR³H) from compounds of general formula VI, whereby radicals X, Y and Z optionally can be further modified. Functional groups that are optionally contained in the intermediate stages, such as carbonyl groups, hydroxy groups or amino groups, can be protected in the meantime with protective groups according to known processes.

Below, examples of ring systems according to the invention corresponding to general formula A are indicated:

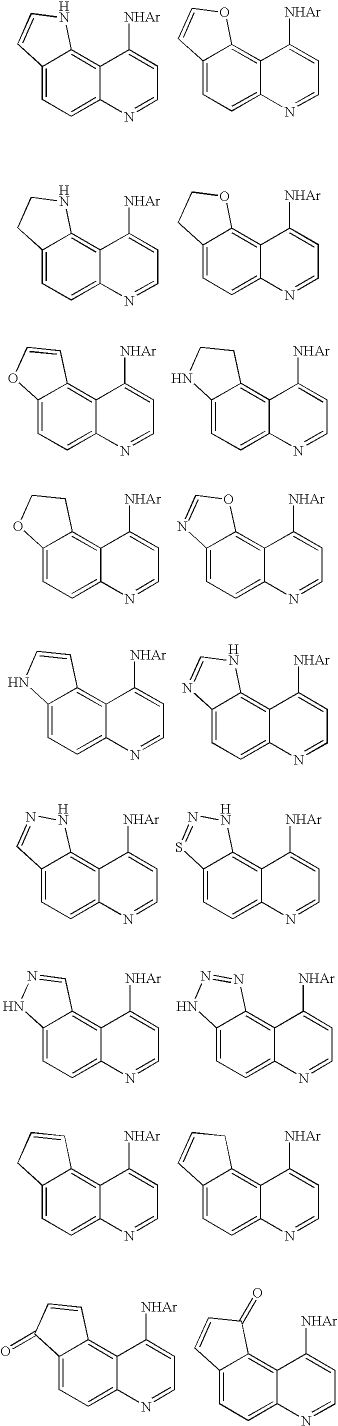

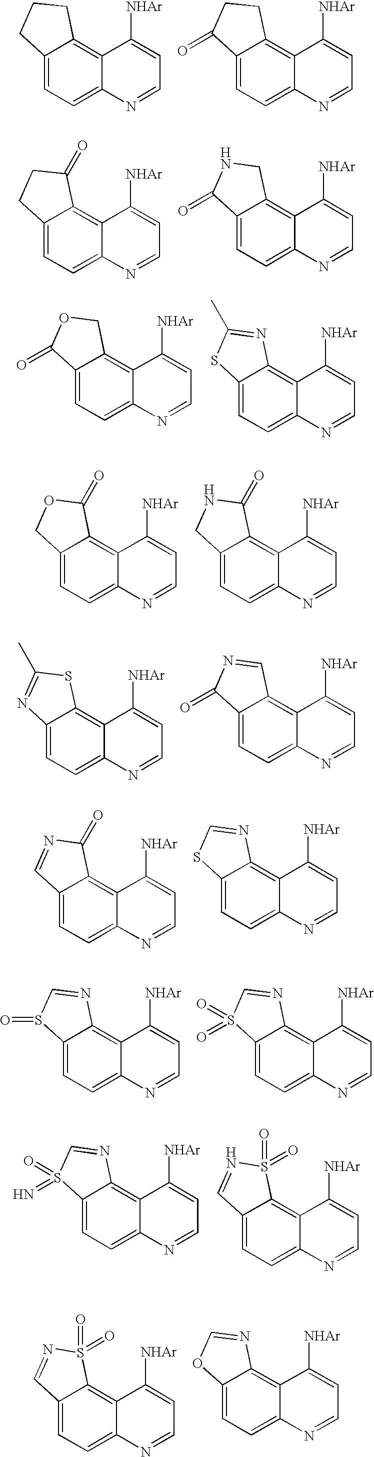

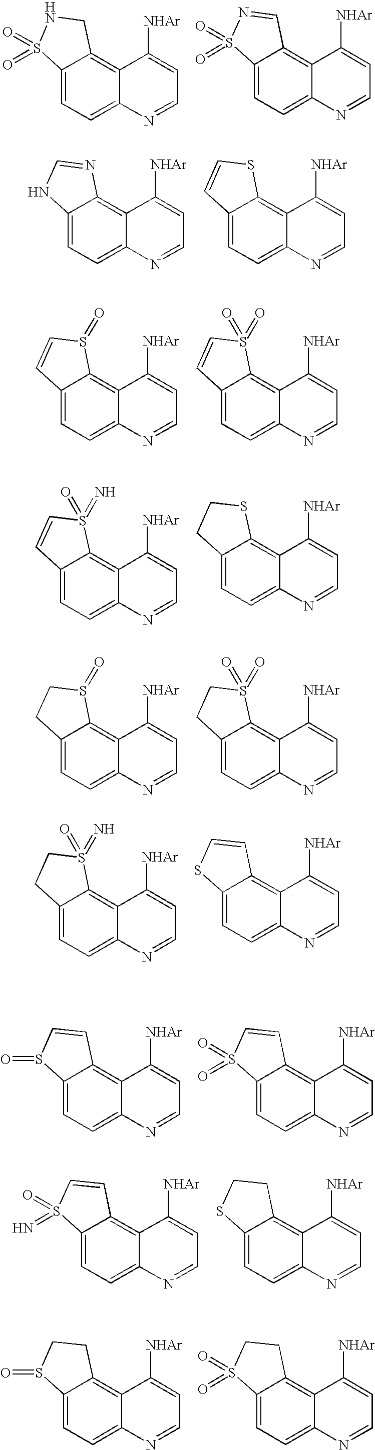

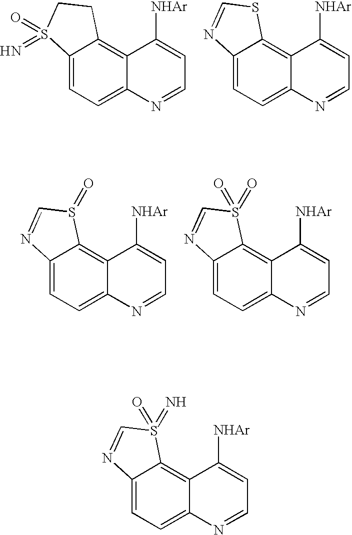

Corresponding to general formula A, instead of —N═ and —NH in the above-mentioned examples, —NR⁴— can also be in the five-membered heterocyclic compound, whereby R⁴ is, for example, C₁-C₁₀-alkyl or C₁-C₁₀-alkanoyl. In the case of —N═, the double bond that originates from N would then be unnecessary.

If X, Y and/or Z in the five-membered ring is carbon, the latter can also be substituted in one or more places, for example they can have alkyl as a radical.

If X, Y, and Z, independently of one another, mean one, two or three N, the understanding holds true that
1. the skeleton in partial grouping X—Y-Z is not N—CH—N, CH—N—N or N—N—N, and
2. X is not NH, if Y and Z are in each case CH at the same time.

The understanding preferably holds true that if X, Y, and Z, independently of one another, mean one, two or three N,
1. the skeleton in partial grouping X—Y-Z is not N—N—CH, N—CH—N, CH—N—N or N—N—N, and
2. X is not NH, if Y and Z are in each case CH at the same time.

### EXAMPLE 1

### Production of 4-Methyl-3-(thieno[3,2-f]quinolin-9-ylamino)-phenol

### EXAMPLE 1a

### Production of 2-(Benzo[b]thiophen-5-ylaminomethylene)-malonic acid diethyl ester

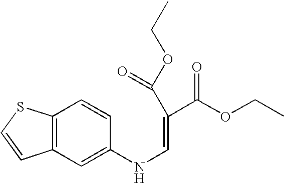

A solution of 540 mg of benzo[b]thiophen-5-ylamine in 5 ml of diethylethoxymethylene malonate is stirred for 1.5 hours at 130° C. Then, the reaction mixture is diluted with ethyl acetate. It is washed with saturated aqueous sodium chloride solution, dried on sodium sulfate, and concentrated by evaporation in a vacuum. The crude product is purified by column chromatography on silica gel with a mixture that consists of hexane/ethyl acetate. 1.88 g of product is obtained.

¹H-NMR (CDCl₃): δ=1.30-1.45 (6H); 4.20-4.38 (4H); 7.16 (1H); 7.30 (1H); 7.51 (1H); 7.58 (1H); 7.86 (1H); 8.60 (1H), 11.12 (1H) ppm.

### EXAMPLE 1b

### Production of 9-Oxo-6,9-dihydro-thieno[3,2-f]quinoline-8-carboxylic acid ethyl ester

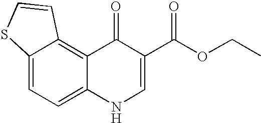

A solution of 315 mg of the compound, described under 1a, in 2 ml of diphenyl ether is stirred for 35 minutes at 240° C. After cooling, it is mixed with cyclohexane, and stirring is continued for one hour at 23° C. The precipitated product is suctioned off and recrystallized from a mixture of dichloromethane and methanol (95:5). 159 mg of product is obtained.

¹H-NMR (d6-DMSO): δ=1.30 (3H); 4.23 (2H); 7.61 (1H); 8.02 (1H); 8.34 (1H); 8.56 (1H); 8.94 (1H); 12.50 (1H) ppm.

### EXAMPLE 1c

### Production of 9-Oxo-6,9-dihydro-thieno[3,2-f]quinoline-8-carboxylic acid

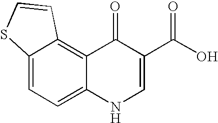

A solution of 500 mg of sodium hydroxide in water is added to a solution of 1 g of the compound, described under Example 1b, in 15 ml of ethanol. It is refluxed for 2 hours. After cooling, it is acidified with 2N hydrochloric acid. Then, stirring is continued for one hour at 23° C. Then, it is suctioned off. 902 mg of product is obtained.

¹H-NMR (d6-DMSO): δ=7.80 (1H); 8.18 (1H); 8.53 (1H); 8.84 (1H); 8.96 (1H); 13.67 (1H); 15.93 (1H) ppm.

### Example 1d

### Production of 6H-Thieno[3,2-f]quinolin-9-one

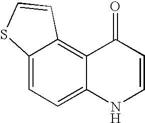

A solution of 100 mg of the compound, described under Example 1c, in 3 ml of diphenyl ether is stirred for 1 hour at 270° C. After cooling, it is diluted with cyclohexane, and stirring is continued for 8 hours at 23° C. It is filtered, and 74 mg of product is obtained.

¹H-NMR (d6-DMSO): δ=6.19 (1H); 7.55 (1H); 7.90-8.03 (2H); 8.26 (1H); 8.94 (1H); 11.99 (1H) ppm.

### EXAMPLE 1e

### Production of 9-Chlorothieno[3,2-f]quinoline

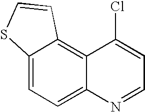

A solution of 150 mg of the compound, described under Example 1d, in 1.5 ml of thionyl chloride is mixed with one drop of N,N-dimethylformamide and then stirred for one hour at 100° C. Then, the reaction mixture is concentrated by evaporation in a vacuum. It is dissolved 3 times in toluene and concentrated by evaporation in a vacuum. Then, the product is stirred for 20 minutes with 2N sodium hydroxide solution. It is suctioned off, the residue is washed with water and dried in a vacuum at 50° C. 138 mg of product is obtained.

¹H-NMR (d6-DMSO): δ=7.85 (1H); 8.00 (1H); 8.12 (1H); 8.46 (1H); 8.76-8.92 (2H) ppm.

### EXAMPLE 1f

### Production of 4-Methyl-3-(thieno[3,2-f]quinolin-9-ylamino)-phenol

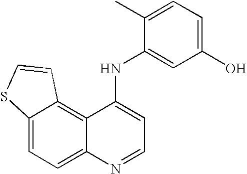

A solution of 130 mg of the compound that is described under Example 1e as well as 85 mg of 3-hydroxy-6-methylaniline in 3 ml of acetonitrile is heated in a sealing tube to 160° C. It is left for 24 hours at this temperature, then allowed to cool, and the reaction mixture is concentrated by evaporation in a vacuum. It is chromatographed on silica gel with a mixture that consists of hexane/ethyl acetate. 54 mg of product is obtained.

¹H-NMR (d6-DMSO): δ=2.08 (3H); 6.47 (1H); 6.50-6.61 (2H); 7.10 (1H); 7.85 (1H); 7.96 (1H); 8.10 (1H); 8.29 (1H); 8.40 (1H); 8.54 (1H); 9.20 (1H) ppm.

### EXAMPLE 2

### Production of 4-Methyl-3-(2-methyl-thiazolo[4,5-f]quinolin-9-ylamino)-phenol

### EXAMPLE 2a

### Production of 2-[(2-Methylbenzothiazol-5-ylamino)-methylene]-malonic acid-diethyl ester

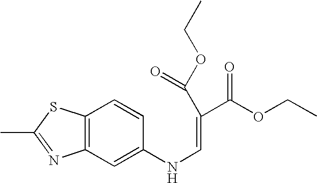

Analogously to Example 1a, 1.31 g of product is obtained from 1 g of 5-amino-2-methylbenzothiazole in diethylethoxymethylene malonate.

¹H-NMR (CDCl₃): δ=1.30-1.45 (6H); 2.84 (3H); 4.20-4.38 (4H); 7.15 (1H); 7.71 (1H); 7.78 (1H); 8.60 (1H); 11.13 (1H) ppm.

### EXAMPLE 2b

### Production of 2-Methyl-9-oxo-6,9-dihydrothiazolo[4,5-f]quinoline-8-carboxylic acid ethyl ester

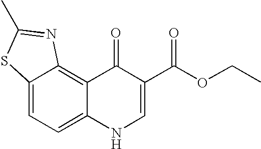

Analogously to Example 1b, 1.09 g of product is obtained from 1.31 g of the compound, described under 2a, in diphenyl ether.

¹H-NMR (CDCl₃): δ=1.46 (3H); 3.00 (3H); 4.50 (2H); 8.01 (1H); 8.13 (1H); 9.28(1H); 13.11 (1H) ppm.

### EXAMPLE 2c

### Production of 2-Methyl-9-oxo-6,9-dihydro-thiazolo[4,5-f]quinoline-8-carboxylic acid

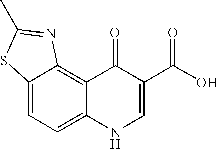

Analogously to Example 1c, 788 mg of product is obtained from 1.05 g of the compound that is described under Example 2b.

¹H-NMR (d6-DMSO): δ=2.92 (3H); 7.80 (1H); 8.53 (1H); 8.91 (1H); 13.55 (1H) ppm.

### EXAMPLE 2d

### Production of 2-Methyl-6H-thiazolo[4,5-f]quinolin-9-one

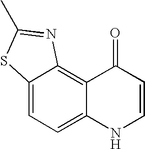

Analogously to Example 1d, 55 mg of product is obtained from 150 mg of the compound, described under Example 2c), in diphenyl ether.

¹H-NMR (d6-DMSO): δ=2.92 (3H); 6.17 (1H); 7.61 (1H); 7.90 (1H); 8.28 (1H); 11.81 (1H) ppm.

### EXAMPLE 2e

### Production of 9-Chloro-2-methyl-thiazolo[4,5-f]quinoline

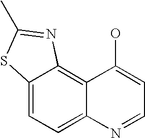

Analogously to Example 1e, 128 mg of product is obtained from 160 mg of the compound, described under Example 2d, in thionyl chloride.

¹H-NMR (d6-DMSO): δ=2.96 (3H); 7.92 (1H); 8.11 (1H); 8.56 (1H); 8.91 (1H) ppm.

### EXAMPLE 2f

### Production of 4-Methyl-3-(2-methyl-thiazolo[4,5-f]quinolin-9-yl-amino)-phenol

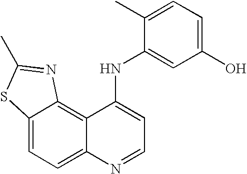

Analogously to Example 1f, 41 mg of product is obtained from 50 mg of the compound that is described under Example 2e as well as 32 mg of 3-hydroxy-6-methylaniline in acetonitrile.

¹H-NMR (d6-DMSO): δ=2.24 (3H); 3.00 (3H); 6.60 (3H); 6.92 (1H); 6.99 (1H); 7.19 (1H); 7.88 (1H); 8.32 (1H); 8.51 (1H); 9.40 (1H); 10.95 (1H) ppm.

### EXAMPLE 3

### Production of 4-Methyl-3-(thieno[2,3-f]quinolin-9-ylamino)-phenol

### EXAMPLE 3a

### Production of 2-(Benzo[b]thiophen-6-ylaminomethylene)malonic acid diethyl ester

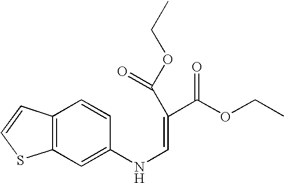

Analogously to Example 1a, 1.31 g of product is obtained from 1 g of 5-amino-2-methylbenzothiazole in diethylethoxymethylene malonate.

¹H-NMR (d6-DMSO) δ=1.20-1.35 (6H); 4.08-4.30 (4H); 7.43 (2H); 7.70 (1H); 7.89 (1H); 8.10 (1H); 8.50 (1H); 10.86 (1H) ppm.

### EXAMPLE 3b

### Production of 9-Oxo-6,9-dihydro-thieno[2,3-f]quinoline-8-carboxylic acid ethyl ester

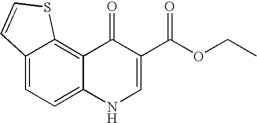

Analogously to Example 1b, 1.09 g of product is obtained from 1.31 g of the compound, described under 3a, in diphenyl ether.

¹H-NMR (d6-DMSO): δ=1.33 (3H); 4.38 (2H); 7.62 (1H); 7.89 (1H); 8.24 (1H); 8.67 (1H); 12.76 (1H) ppm.

### EXAMPLE 3c

### Production of 9-Oxo-6,9-dihydro-thieno[2,3-f]quinoline-8-carboxylic acid

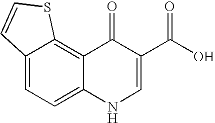

Analogously to Example 2c, 788 mg of product is obtained from 1.05 g of the compound that is described under Example 3b.

¹H-NMR (d6-DMSO): δ=7.72 (1H); 7.84 (1H); 8.04 (1H); 8.41 (1H); 8.98 (1H); 13.78 (1H) ppm.

### EXAMPLE 3d

### Production of 6H-Thieno[2,3-f]quinolin-9-one

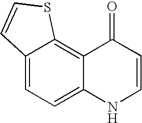

Analogously to Example 1d, 483 mg of product is obtained from 640 mg of the compound, described under Example 3c, in diphenyl ether.

¹H-NMR (d6-DMSO): δ=6.30 (1H); 7.55-7.66 (2H); 7.80 (1H); 8.03 (1H); 8.18 (1H); 12.23 (1H) ppm.

### EXAMPLE 3e

### Production of Trifluoromethanesulfonic acid-thieno[2,3-f]quinolin-9-yl-ester

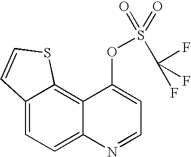

250 μl of trifluoromethanesulfonic acid anhydride is added at 0° C. to a solution of 100 mg of the substance, described under Example 3d; in 2 ml of pyridine. It is allowed to come to 21° C. and stirred for 45 more minutes at this temperature. Then, the reaction mixture is poured onto saturated aqueous sodium chloride solution. It is allowed to stir for 2 more hours and then suctioned off. The residue is purified by column chromatography on silica gel with a mixture that consists of hexane/ethyl acetate. 106 mg of product is obtained.

¹H-NMR (d6-DMSO): δ=6.68 (1H); 7.68 (1H); 7.75 (1H); 7.94 (1H); 8.28-8.40 (1H) ppm.

### EXAMPLE 3f

### Production of 4-Methyl-3-(thieno[2,3-f]quinolin-9-ylamino)phenol

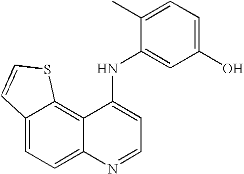

A solution of 100 mg of the compound, described under 3e, and 75 mg of 3-hydroxy-6-methylaniline in 5 ml of acetonitrile is stirred for 24 hours at 50° C. Then, the precipitated reaction product is suctioned off and purified by column chromatography on silica gel with a mixture that consists of hexane/ethyl acetate. 75 mg of product is obtained.

¹H-NMR (d6-DMSO): δ=2.06 (3H); 6.60 (1H); 6.78-6.90 (2H), 7.26 (1H); 7.91 (1H); 8.08 (1H); 8.21 (1H); 8.52-8.65 (2H); 9.38 (1H) ppm.

### EXAMPLE 4

### Production of 3-(3,3-Dioxo-2,3-dihydro-1H-3λ⁶-thieno[3,2-f]quinolin-9-ylamino)-4-methyl-phenol

### EXAMPLE 4a

### Production of 2-[(1,1-Dioxo-2,3-dihydro-1H-1λ⁶-benzo[b]thiophen-5-ylamino)-methylene]malonic acid diethyl ester

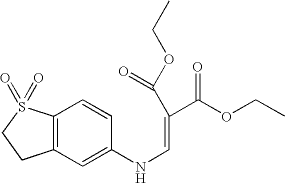

Analogously to Example 1a, 613 mg of product is obtained from 340 mg of 1,1-dioxo-2,3-dihydro-1H-1λ⁶-benzo[b]thiophen-5-ylamine in diethylethoxymethylene malonate.

¹H-NMR (d6-DMSO): δ=1.25 (6H); 3.32 (2H); 3.59 (2H), 4.18 (2H); 7.50 (1H); 7.54 (1H); 7.72 (1H); 8.42 (1H); 10.72 (1H) ppm.

### EXAMPLE 4b

### Production of 3,3,9-Trioxo-2,3,6,9-tetrahydro-1H-3λ⁶-thieno[3,2-f]quinoline-8-carboxylic acid ethyl ester

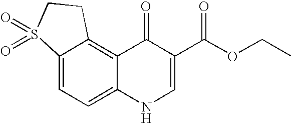

Analogously to Example 1b, 162 mg of product is obtained from 100 mg of the compound, described under 4a, in diphenyl ether.

¹H-NMR (d6-DMSO): δ=1.28 (3H); 3.62 (2H); 3.96 (2H); 4.22 (2H); 7.72 (1H); 7.96 (1H); 8.56 (1H); 12.60 (1H) ppm.

### EXAMPLE 4c

### Production of 3,3,9-Trioxo-2,3,6,9-tetrahydro-1H-3λ⁶-thieno-[3,2-f]quinoline-8-carboxylic acid

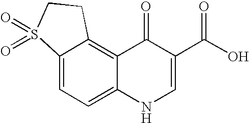

Analogously to Example 1c, 382 mg of product is obtained from 444 mg of the compound that is described under Example 4b.

¹H-NMR (d6-DMSO): δ=3.69 (2H); 4.00 (2H); 7.90 (1H); 8.12 (1H); 8.97 (1H); 13.66 (1H); 14.98 (1H) ppm.

### EXAMPLE 4d

### Production of 3,3-Dioxo-1,2,3,6-tetrahydro-3λ⁶-thieno[3,2-flquinolin-9-one

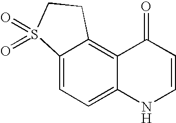

Analogously to Example 1d, 280 mg of product is obtained from 380 mg of the compound, described under Example 4c, in diphenyl ether.

¹H-NMR (d6-DMSO): δ=3.59 (2H); 3.95 (2H); 6.11 (1H); 7.63 (1H); 7.85-8.02 (2H); 12.09 (1H) ppm.

### EXAMPLE 4e

### Production of 9-Chloro-1,2-dihydro-thieno[3,2-f]quinoline 3,3-dioxide

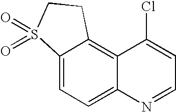

Analogously to Example 1e, 512 mg of product is obtained from 500 mg of the compound, described under Example 4d, in thionyl chloride.

¹H-NMR (d6-DMSO): δ=3.77 (2H); 4.16 (2H); 7.90 (1H); 8.06 (1H); 8.21 (1H); 8.95 (1H) ppm.

### EXAMPLE 4f

### Production of 3-(3,3-Dioxo-2,3-dihydro-1H-3λ⁶-thieno[3,2-f]quinolin-9-ylamino)-4-methyl-phenol

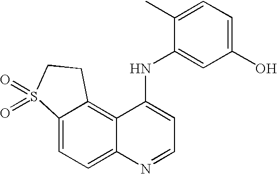

Analogously to Example 1f, 51 mg of product is obtained from 83 mg of the compound that is described under Example 4e as well as 80 mg of 3-hydroxy-6-methylaniline in acetonitrile.

¹H-NMR (d6-DMSO): δ=2.10 (3H); 3.80 (2H); 4.24 (2H); 6.49 (1H); 6.79 (1H); 6.84 (1H); 7.26 (1H); 8.19 (1H); 8.28 (1H); 8.52 (1H); 9.61 (1H); 9.89 (1H) ppm.

### EXAMPLE 5

### Production of 3-(3,3-Dioxo-2,3-dihydro-1H-3λ⁶-thieno[3,2-f]quinolin-9-ylamino)-phenol

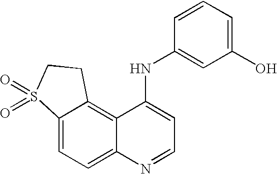

Analogously to Example 4f, 73 mg of product is obtained from 90 mg of the compound that is described under Example 4e as well as 80 mg of 3-aminophenol in acetonitrile.

¹H-NMR (d6-DMSO): δ=3.76 (2H); 4.22 (2H); 6.82 (1H); 6.90 (2H); 7.02 (1H); 8.18 (1H); 8.28 (1H); 8.58 (1H); 9.80 (1H); 9.98 (1H) ppm.

### EXAMPLE 6

### Production of 4-(3,3-Dioxo-2,3-dihydro-1H-3λ⁶-thieno[3,2-f]quinolin-9-ylamino)-3-methyl-phenol

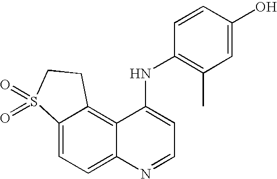

Analogously to Example 4f, 37 mg of product is obtained from 90 mg of the compound that is described under Example 4e as well as 90 mg of 4-amino-3-methyl-phenol in acetonitrile.

¹H-NMR (d6-DMSO): δ=3.78 (2H); 4.26 (2H); 6.34(1H); 6.80 (1H); 6.88 (1H); 7.12 (1H); 8.15 (1H); 8.28 (1H); 8.48 (1H); 9.43 (1H); 9.84 (1H) ppm.

### EXAMPLE 7

### Production of 2-(3,3-Dioxo-2,3-dihydro-1H-3λ⁶-thieno[3,2-f]quinolin-9-ylamino)-phenol

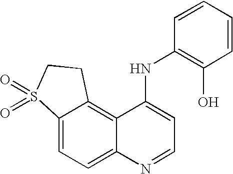

Analogously to Example 4f, 59 mg of product is obtained from 90 mg of the compound that is described under Example 4e as well as 80 mg of 2-aminophenol in acetonitrile.

¹H-NMR (d6-DMSO): δ=3.80 (2H); 4.22 (2H); 6.52 (1H); 6.99 (1H); 7.12 (1H); 7.28-7.40 (2H); 8.16 (1H); 8.28 (1H); 8.54 (1H); 9.48 (1H); 10.20 (1H) ppm.

### EXAMPLE 8

### Production of 4-(3,3-Dioxo-2,3-dihydro-1H-3λ⁶-thieno[3,2-f]quinolin-9-ylamino)-phenol

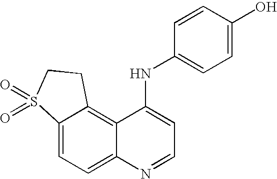

Analogously to Example 4f, 47 mg of product is obtained from 90 mg of the compound, described under Example 4e, as well as 80 mg of 4-aminophenol in acetonitrile.

¹H-NMR (d6-DMSO): δ=3.74 (2H); 4.22 (2H); 6.78 (1H); 6.95 (2H); 7.28 (2H); 8.12 (1H); 8.24 (1H); 8.50 (1H); 9.65 (1H); 9.91 (1H) ppm.

### EXAMPLE 9

### Production of [3-(3,3-Dioxo-2,3-dihydro-1H-3λ⁶-thieno[3,2-f]quinolin-9-ylamino)phenyl]-methanol

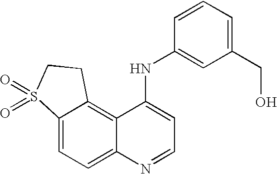

Analogously to Example 4f, 88 mg of product is obtained from 90 mg of the compound that is described under Example 4e as well as 90 mg of 3-aminobenzyl alcohol in acetonitrile.

¹H-NMR (d6-DMSO): δ=3.76 (2H); 4.25 (2H); 4.58 (2H); 7.00 (1H); 7.35 (2H); 7.46 (1H); 7.53 (1H); 8.18 (1H); 8.39 (1H); 8.59 (1H); 9.99 (1H) ppm.

### EXAMPLE 10

### Production of 3-(3,3-Dioxo-2,3-dihydro-1H-3λ⁶-thieno[3,2-f]quinolin-9-ylamino)-benzoic acid

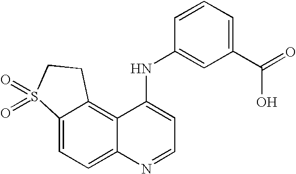

Analogously to Example 4f, 65 mg of product is obtained from 90 mg of the compound that is described under Example 4e as well as 100 mg of 3-aminobenzoic acid in acetonitrile.

¹H-NMR (d6-DMSO): δ=3.73 (2H); 4.28 (2H); 7.06 (1H); 7.66-7.83 (2H); 7.95 (1); 8.06 (1H); 8.20 (1H); 8.30 (1H); 8.61 (1H); 10.00 (1H); 13.28 (1H) ppm.

### EXAMPLE 11

### Production of 3-(3,3-Dioxo-2,3-dihydro-1H-3λ⁶-thieno[3,2-f]quinolin-9-ylamino)-benzamide

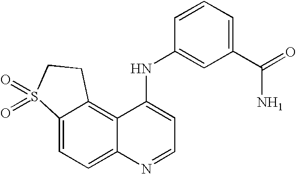

Analogously to Example 4f, 54 mg of product is obtained from 90 mg of the compound that is described under Example 4e as well as 100 mg of 3-aminobenzarnidine.

¹H-NMR (d6-DMSO): δ=3.78 (2H); 4.28 (2H); 7.03 (1H); 7.52 (1H); 7.67 (2H); 7.90 (1H); 7.99 (1H); 8.12 (1H); 8.21 (1H); 8.29 (1H); 8.60 (1H); 9.98 (1H) ppm.

### EXAMPLE 12

### Production of (3,3-Dioxo-2,3-dihydro-1H-3λ⁶-thieno[3,2-f]quinolin-9-yl)-(3-methoxyphenyl]amine

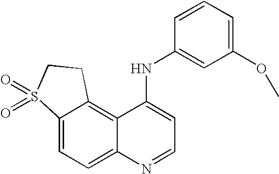

Analogously to Example 4f, 106 mg of product is obtained from 90 mg of the compound that is described under Example 4e as well as 100 mg of 3-methoxyphenylamine in acetonitrile.

¹H-NMR (d6-DMSO): δ=3.70-3.88 (5H); 4.26 (2H); 6.95-7.16 (4H); 7.50 (1H); 8.19 (1H); 8.28 (1H); 8.58 (1H); 9.90 (1H) ppm.

### EXAMPLE 13

### Production of N-[3-(3,3-Dioxo-2,3-dihydro-1H-3λ⁶-thieno[3,2-f]quinolin-9-ylamino)-phenyl]-acetamide

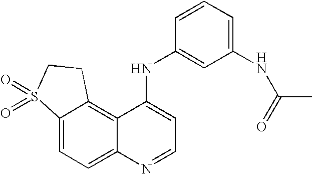

Analogously to Example 4f, 146 mg of product is obtained from 150 mg of the compound that is described under Example 4e as well as 180 mg of N-(3-aminophenyl)-acetamide in acetonitrile.

¹H-NMR (d6-DMSO): δ=2.08 (3H); 3.74 (2H); 4.23 (2H); 7.02 (1H); 7.16 (1H); 7.40-7.55 (2H); 7.96 (1H); 8.18 (1H); 8.28 (1H); 8.59 (1H); 9.90 (1H); 10.32 (1H) ppm.

### EXAMPLE 14

### Production of 3-(3,3-Dioxo-2,3-dihydro-1H-3λ⁶-thieno [3,2-f]quinolin-9-ylamino)-5-methoxyphenol

### EXAMPLE 14a

### Production of Trifluoromethanesulfonic acid-3,3-dioxo-2,3-dihydro-1H-3λ⁶-thieno[3,2-f]quinolin-9-yl-ester

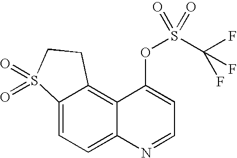

Analogously to Example 3e, 348 mg of product is obtained from 300 mg of the compound that is described under Example 4d and 645 μl of trifluoromethanesulfonic acid anhydride in pyridine.

¹H-NMR (d6-DMSO): δ=3.78 (2H); 3.90 (2H); 7.86 (1H); 8.17 (1H); 8.30 (1H); 9.20 (1H) ppm.

### EXAMPLE 14b

### Production of 3-(3,3-Dioxo-2,3-dihydro-1H-3λ⁶-thieno[3,2-f]quinolin-9-ylamino)-5-methoxyphenol

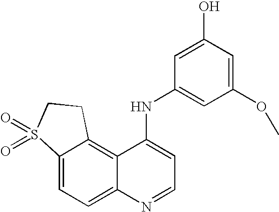

Analogously to Example 3f, 59 mg of product is obtained from 100 mg of the compound that is described under 14a and 85 mg of 3-amino-5-methoxyphenol in acetonitrile.

¹H-NMR (d6-DMSQ): δ=3.60-3.75 (5H); 4.07 (2H); 6.06 (1H); 6.28 (2H); 7.35 (1H); 7.89 (1H); 8.00 (1H); 8.27 (1H); 8.66 (1H); 9.50 (1H) ppm.

### EXAMPLE 15

### Production of 5-(3,3-Dioxo-2,3-dihydro-1H-3λ⁶-thieno[3,2-f]quinolin-9-ylamino)-2-methylphenol

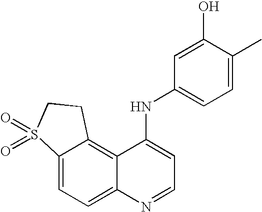

Analogously to Example 14b, 58 mg of product is obtained from 120 mg of the compound that is described under Example 14a and 80 mg of 5-amino-2-methylphenol in acetonitrile.

¹H-NMR (d6-DMSO): δ=2.10 (3H); 3.67 (2H); 4.10 (2H); 6.62 (1H); 6.74 (1H); 7.06 (1H); 7.17 (1H); 7.87 (1H); 7.98 (1H); 8.18 (1H); 8.59 (1H); 9.40 (1H) ppm.

### EXAMPLE 16

### Production of 3-(3,3-Dioxo-2,3-dihydro-1H-3λ⁶-thieno[3,2-f]quinolin-9-ylamino)-2-methylphenol

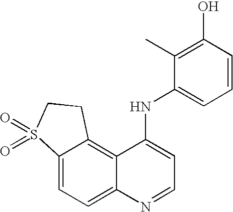

Analogously to Example 14b, 32 mg of product is obtained from 120 mg of the compound that is described under Example 14a and 80 mg of 3-amino-2-methylphenol in acetonitrile.

¹H-NMR (d6-DMSO): δ=2.00 (3H); 3.75 (2H); 4.27 (2H); 6.35 (1H); 6.71 (1H); 7.00 (1H); 7.18 (1H); 8.48 (1H) ppm.

Analogously to the process that is described under Example 14b, the examples that are shown in the following table are produced from the compound that is described under 14a and the respective aniline derivative:

| Example | Structure | Name | δ 1H-NMR ppm |
|---|---|---|---|
| 17 | 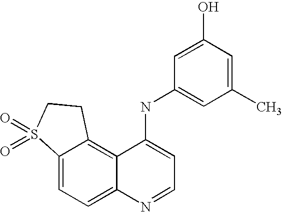 | 3-(3,3-Dioxo-2,3-dihydro- 1H-3λ⁶-thieno[3,2- f]quinolin-9-ylamino)-5- methyl-phenol | (d6-DMSO, 400 MHz): 2.16 (3H); 3.61 (2H); 4.03 (2H); 6.28 (1H); 6.47 (2H); 7.23 (1H); 7.84 (1H); 7.93 (1H); 8.18 (1H); 8.60 (1H); 9.33 (1H) |
| 18 | 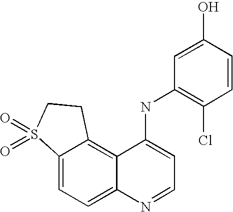 | 4-Chloro 3-(3,3-dioxo- 2,3-dihydro-1H-3λ⁶- thieno[3,2-f]quinolin-9- ylamino)-5-methyl-phenol | (d6-DMSO, 1 drop of DCl; 300 MHz): 3.74 (2H); 4.22 (2H); 6.48 (1H); 6.91 (1H); 7.02 (1H); 7.46 (1H); 8.25 (2H); 8.57 (1H) |
| 19 | 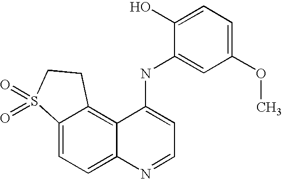 | 2-(3,3-Dioxo-2,3-dihydro- 1H-3λ⁶-thieno[3,2- f]quinolin-9-ylamino)-4- methoxy-phenol | (d6-DMSO, 1 drop of DCl; 300 MHz): 3.67 (3H); 3.71 (2H); 4.23 (2H); 6.52 (1H); 6.86 (1H); 6.97-7.06 (2H); 8.21 (2H); 8.50 (1H) |
| 20 | 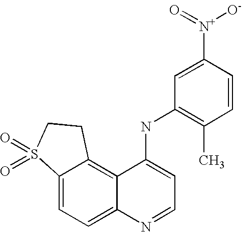 | (3,3-Dioxo-2,3-dihydro- 1H-3λ⁶-thieno[3,2-f]- quinolin-9-yl)-(2- methyl-5-nitro-phenyl)- amine | (d6-DMSO, 1 drop of DCl; 300 MHz): 2.29 (3H); 3.72 (2H); 4.33 (2H); 6.41 (1H); 7.71 (1H); 8.15-8.37 (4H); 8.52 (1H) |
| 21 | 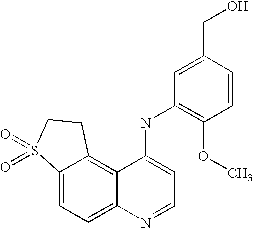 | [3-(3,3-Dioxo-2,3- dihydro-1H-3λ⁶- thieno[3,2-f]quinolin-9- ylamino)-4-methoxy- phenyl]-methanol | (d6-DMSO, 300 MHz): 3.71-3.82 (5H); 4.16 (2H); 4.47 (2H); 6.51 (1H); 7.21 (1H); 7.35 (2H); 8.01 (1H); 8.22 (1H); 8.50 (1H); 9.40 (1H) |
| 22 | 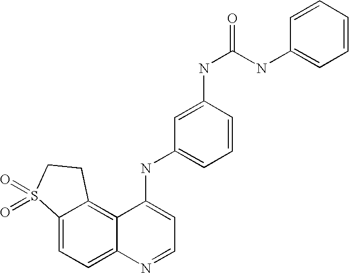 | 1-[3-(3,3-Dioxo-2,3- dihydro-1H-3λ⁶- thieno[3,2-f]quinolin- 9-ylamino)-phenyl]-3- phenyl-urea | (d6-DMSO, 400 MHz): 3.77 (2H); 4.21 (2H); 6.98 (1H); 7.08 (2H); 7.29 (3H); 7.42-7.52 (3H); 7.87 (1H); 8.05 (1H); 8.28 (1H); 8.61 (1H); 8.74 (1H); 8.95 (1H); 9.76 (1H) |
| 23 | 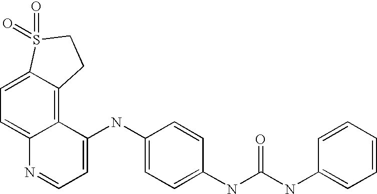 | 1-[4-(3,3-Dioxo-2,3- dihydro-1H-3λ⁶- thieno[3,2-f]quinolin- 9-ylamino)-phenyl]-3- phenyl-urea | (d6-DMSO, 400 MHz): 3.72 (2H); 4.17 (2H); 6.86 (1H); 6.95 (1H); 7.25 (2H); 7.35 (2H); 7.43 (2H); 7.63 (2H); 7.99 (1H); 8.22 (1H); 8.50 (1H); 8.70 (1H); 8.90 (1H); 9.61 (1H) |
| 24 | 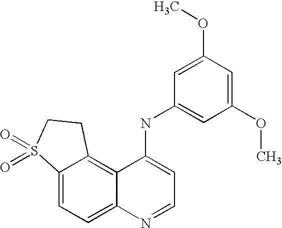 | (3,5-Dimethoxy-phenyl)- (3,3-dioxo- 2,3-dihydro-1H-3λ⁶- thieno[3,2-f]quinolin-9- yl)-amine | (d6-DMSO, 300 MHz): 3.68-3.84 (8H); 4.16 (2H); 6.52 (1H); 6.60 (2H); 7.10 (1H); 8.01 (1H); 8.23 (1H); 8.56 (1H); 9.60 (1H) |
| 25 | 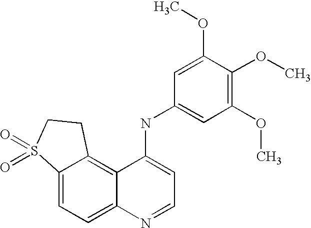 | (3,3-Dioxo-2,3-dihydro- 1H-3λ⁶-thieno[3,2-f]- quinolin-9-yl)-(3,4, 5-trimethoxy-phenyl)- amine | (d6-DMSO, 400 MHz): 3.66-3.82 (11H); 4.17 (2H); 6.78 (2H); 7.05 (1H); 8.00 (1H); 8.21 (1H); 8.51 (1H); 9.57 (1H) |

The anilines that are required for the production of Examples 22 and 23 are produced as follows:

1-(3-Amino-phenyl)-3-phenyl-urea (for Example 22)

3 g of 3-nitroaniline is dissolved in 50 ml of dichloromethane. 3.5 ml of phenyl isocyanate is added, and stirring is allowed to continue for 22 hours at 23° C. Then, the precipitated reaction product is filtered off. The crude product is dissolved in a mixture that consists of 30 ml of tetrahydrofuran and 16 ml of ethanol, 150 mg of palladium/carbon (10%) is added under hydrogen, and it is hydrogenated at normal pressure for 1.5 hours. Then, the reaction mixture is filtered on Celite. It is concentrated by evaporation in a vacuum, and the crude product that is obtained is crystallized from diisopropyl ether. 2.3 g of product is obtained.

1-(4-Amino-phenyl)-3-phenyl-urea (for Example 23)

The production is carried out analogously to the above-mentioned process for 1-(3-amino-phenyl)-3-phenyl-urea, whereby 4-nitroaniline is used as a starting material.

### EXAMPLE 26

### Production of N³-(3,3-Dioxo-2,3-dihydro-1H-3λ⁶-thieno[3,2-f]quinolin-9-yl)-4-methyl-phenyl-1,3-diamine

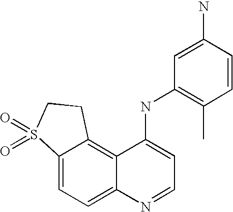

100 mg of the compound that is described under Example 20 is dissolved in a mixture that consists of 5 ml of tetrahydrofuran and 3 ml of ethanol. 20 mg of palladium/carbon (10%) is added, placed under hydrogen and hydrogenated at normal pressure for 4.5 hours. Then, the reaction mixture is filtered on Celite. It is concentrated by evaporation in a vacuum, and the crude product that is obtained is purified by column chromatography on silica gel. 91 mg of product is obtained.

(d6-DMSO, 1 drop of DCl; 400 MHz): δ=2:18 (3H); 3.70 (2H); 4.27 (2H); 6.31 (1H); 7.41 (1H); 7.46 (1H); 7.54 (1H); 8.23 (2H); 8.50 (1H) ppm.

### EXAMPLE 27

### Production of 1-[3-(3,3-Dioxo-2,3-dihydro-1H-3λ⁶-thieno[3,2-f]quinolin-9-ylamino)-4-methyl-phenyl]-3-phenyl-urea

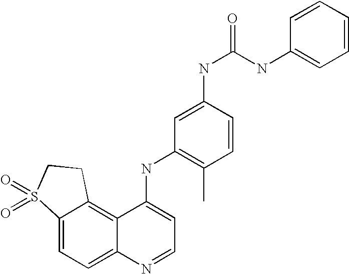

22 mg of the compound that is described under Example 26 is dissolved in 2 ml of dichloromethane. 12 μl of phenyl isocyanate is added and allowed to stir for 16 more hours at 23° C. Then, it is diluted with some diisopropyl ether. The precipitated reaction product is filtered out, and the crude product is then stirred with diisopropyl ether. 20 mg of product is obtained. (d6-DMSO, 1 drop of DCl; 400 MHz): δ=2.18 (3H); 3.72 (2H); 4.22 (2H); 6.34 (1H); 6.87 (1H); 7.18 (2H); 7.30 (1H); 7.36 (3H); 7.54 (1H); 8.20 (2H); 8.46 (1H) ppm.

Biological Tests of the Compounds

Test System for EphB4

A mixture that consists of 20 ng/ml of recombinant EphB4 kinase (ProQinase GmbH, Freiburg, Germany), 2.67 μg/ml of polyGluAlaTyr, 2 μM of ATP, 25 mmol of HEPES (pH 7.3), 5 mmol of MgCl₂, 1 mmol of MnCl₂, 2 mmol of DTT, 0.1 mmol of NaVO₄, 1% (v/v) of glycerol, 0.02% NP40, EDTA-free protease inhibitors (Complete Roche Company, 1 tablet in 50 ml) is incubated for 10 minutes at 20° C. Test substances are dissolved in 100% DMSO and introduced in a 0.017× volume before the reaction begins. 60 minutes after 1.7× volume of a solution of 50 mmol of Hepes, pH 7.0, 0.2% BSA, 0.14 μg/ml of PT66-Europium, 3.84 μg/ml of SA-XL665, 75 mmol of EDTA is added, the batch is measured in a Discovery HTRF measuring device of the PerkinElmer Company. Among others, the following compounds inhibit the EphB4 kinase with an IC₅₀, which is smaller than 25 μM: Examples 1, 2, 3, 4, 5 and 15 of the description according to the invention. The IC₅₀ of the compound according to Example 2 is 270 nM.

This illustrates that the substances according to the invention inhibit receptor tyrosine kinases, in particular Eph receptors and here in particular EphB4.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 102004063223.5, filed Dec. 22, 2005 and U.S. Provisional Application Ser. No. 60/641,733, filed Jan. 7, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Quinoline derivative with general formula A: 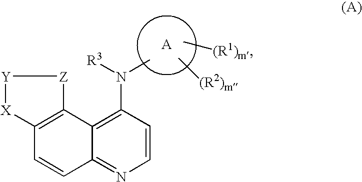
whereby
A is selected from the group that comprises —C₆-C₁₂-aryl, —C₅-C₁₈-heteroaryl, —C₃-C₁₂cycloalkyl and —C₃-C₁₂-heterocycloalkyl,
R¹ and R² are the same or different and are selected in one or more places, independently of one another, from the group that comprises hydrogen, hydroxy, halogen, nitro, cyano, —C₁-C₆-alkyl, —C₁-C₄-hydroxyalkyl, —C₂-C₆-alkenyl, —C₂-C₆-alkinyl, —C₃-C₁₀-cycloalkyl, —C₃-C₁₂-heterocycloalkyl, —C₆-C₁₂-aryl, —C₅-C₁₈-heteroaryl, —C₁-C₆-alkoxy, —C₁-C₆-alkoxy-C₁-C₆-alkoxy, —C₁-C₆-alkoxy-C₁-C₆-alkyl, —C₁-C₆-alkoxy-C₁-C₆-alkoxy-C₁-C₆-alkyl, —(CH₂)ₙ—C₆-C₁₂-aryl, —(CH₂)ₙ—C₅-C₁₈-heteroaryl, —(CH₂)ₙ—C₃-C ₁₀-cycloalkyl, —(CH₂)ₙ—C₃-C₁₂-heterocycloalkyl, -phenylene-(CH₂)ₚ—R⁶, —(CH₂)ₚPO₃(R⁶)₂, —(CH₂)ₚ—NR⁵R⁶, —(CH₂)ₚ—NR⁴COR⁵, —(CH₂)ₚ—NR⁴CSR⁵, —(CH₂)ₚ—NR⁴S(O)R⁵, —(CH₂)ₚ—NR⁴S(O)₂R⁵, —(CH₂)ₚ—NR⁴CONR⁵R⁶, —(CH₂)ₚ-NR⁴COOR⁵, —(CH₂)ₚ—NR⁴C(NH)NR⁵R⁶, —(CH₂)ₚ—NR⁴CSNR⁵R⁶, —(CH₂)ₚ—NR⁴S(O)NR⁵R⁶, —(CH₂)ₚ—NR⁴S(O)₂NR⁵R⁶, —(CH₂)ₚ—COR⁵, —(CH₂)ₚ—CSR⁵, —(CH₂)ₚ—S(O)R⁵, —(CH₂)ₚ—S(O)(NH)R⁵, —(CH₂)ₚ—S(O)₂R⁵, —(CH₂)ₚ—S(O)₂NR⁵R⁶, —(CH₂)ₚ—SO₂OR⁵, —(CH₂)ₚ—CO₂R⁵, —(CH₂)ₚ—CONR⁵R⁶, —(CH₂)ₚ—CSNR⁵R⁶, —OR⁵, —(CH₂)ₚ—SR⁵ and —CR⁵(OH)—R⁶, whereby —C₁-C₆-alkyl, —C₂-C₆-alkenyl, —C₂-C₆-alkinyl, —C₃-C₁₀-cycloalkyl, —C₃-C₁₂-heterocycloalkyl, —C₆-C₁₂-aryl, —C₅-C₁₈-heteroaryl and/or —C₁-C₆-alkoxy are unsubstituted or are substituted in one or more places, independently of one another, with hydroxy, halogen, nitro, cyano, phenyl, —NR⁵R⁶, alkyl and/or —OR⁵, whereby the carbon skeleton of the —C₃-C₁₀-cycloalkyl and the —C₁-C₁₀-alkyl can contain nitrogen, oxygen or sulfur atoms and/or C═O groups and/or one or more double bonds in one or more places, independently of one another, and/or R¹ and R² optionally form a bridge with one another that consists of 3-10 methylene units, whereby up to two methylene units are optionally replaced by O, S and/or —NR⁴,
X, Y, Z are the same or different and are selected independently of one another from the group that comprises —CR³═, —CR³R⁴—, —C(O)—, —N═, —S—, —O—, —NR³—, —S(O)₂—, —S(O)— and —S(O)NH— and single or double bonds are found between X, Y and Z,
R³ is hydrogen, —C₁-C₀-alkyl or —C₁-C₁₀-alkanoyl,
R⁴ is hydrogen or —C₁-C₁₀-alkyl,
R⁵ and R⁶ are the same or different and are selected, independently of one another, from the group that comprises hydrogen, —C₁-C₁₀-alkyl, —C₂-C₁₀-alkenyl, —C₂-C₁₀-alkinyl, —C₁-C₆-alkoxy, —C₃-C₁₀-cycloalkyl, —C₃-C₁₂-heterocycloalkyl, —C₆C₁₂-aryl and —C₅-C₁₈-heteroaryl, whereby —C₁-C₁₀-alkyl, —C₂-C₁₀-alkenyl, —C₂-C₁₀-alkinyl, —C₁-C₆-alkoxy, —C₃-C₁₀-cycloalkyl, —C₃-C₁₂-heterocycloalkyl, —C₆-C₁₂-aryl and/or —C₅-C₁₈-heteroaryl are unsubstituted or [are substituted] in one or more places, independently of one another, with hydroxy, halogen, cyano, nitro, —OR⁷, —NR⁷R⁸,
—C(O)NR⁷R⁸, —C(O)OR⁷ and/or —C₁-C₆-alkyl, whereby —C₁-C₆-alkyl is unsubstituted or [is substituted] in one or more places, independently of one another, with halogen, hydroxy, cyano, —NR⁷R⁸, —OR⁷ and/or phenyl; and/or R⁵ and R⁶ optionally form a bridge with one another that consists of 3-10 methylene units, whereby up to two methylene units optionally are replaced with O, S and/or NR⁴,
R⁷ and R⁸ are the same or different and are selected, independently of one another, from the group that comprises hydrogen, —C₁-C₄-alkyl, —C₆-C₁₂-aryl and —C₅-C₁₈-heteroaryl, whereby alkyl, aryl, or heteroaryl is unsubstituted or [is substituted] in one or more places, independently of one another, with halogen and/or alkoxy, or R⁷ and R⁸ optionally form a bridge with one another that consists of 3-10 methylene units, whereby up to two methylene units optionally are replaced with O, S and/or —NR⁴;
m′, m″=0-4, independently of one another,
n=1-6,
p=0-6, as well as
their N-oxides, solvates, hydrates, stereoisomers, diastereomers, enantiomers and salts, provided that if X, Y, and Z, independently of one another, mean one, two or three N,
1. The skeleton in partial grouping X—Y-Z is not N—CH—N, CH—N—N or N—N—N, and
2. X is not NH, if Y and Z are simultaneously CH in each case.

2. Quinoline derivative according to claim 1, characterized in that if X, Y, and Z, independently of one another, mean one, two or three N,
1. The skeleton in partial grouping X—Y-Z is not N—N—CH, N—CH—N, CH—N—N or N—N—N, and
2. X is not NH, if Y and Z are simultaneously CH in each case.

3. Quinoline derivative according to claim 1, wherein A is phenyl.

4. Quinoline derivative according to claim 3, wherein
R¹ and R² are the same or different and are selected in one or more places, independently of one another, from the group that comprises hydrogen, hydroxy, halogen, nitro, cyano, —C₁-C₆-alkyl, —C₁-C₄-hydroxyalkyl, —C₆-C₁₂-aryl, —C₁-C₆-alkoxy, —NR⁵R⁶, —NR⁴COR⁵, —NR⁴S(O)R⁵, —NR⁴S(O)₂R⁵, —NR⁴CONR⁵R⁶, —NR⁴S(O)NR⁵R⁶, —NR⁴S(O)₂NR⁵R⁶, —COR⁵, COOR⁵, —S(O)R⁵, —S(O)(NH)R⁵, —S(O)₂R⁵, —S(O)₂NR⁵R⁶, —CO₂R⁵, —CONR⁵R⁶, —OR⁵ and —CR⁵(OH)—R⁶, and
m′, m″=0-3, independently of one another.

5. Quinoline derivative according to claim 1, wherein X, Y and Z, independently of one another, are selected from the group that comprises —CR⁴═,
—CR⁴R⁵, —C(O)—, —N═, —S—, —O—, —NR⁴—, —S(O)₂—, —S(O)— and —S(O)NH—, whereby N, S or O do not occur in several places in the ring.

6. Quinoline derivative according to claim 1, wherein X, Y and Z stand for —S(O)₂—, —S—, —NH—, —CH═, —C(CH₃)═, and/or —CH₂—.

7. Quinoline derivative according to claim 1, wherein the skeleton in partial grouping X—Y-Z is selected from the group that comprises —S—CH═CH—, —S—C(C₁-C₆-alkyl)═N—, —S(O)₂—CH₂-CH₂— and —CH═CH—S—.

8. Quinoline derivative according to claim 1, wherein R³ is hydrogen.

9. Quinoline derivative according to claim 1, wherein
A is phenyl,
R¹ and R² are the same or different and are selected in one or more places, independently of one another, from the group that comprises hydrogen, hydroxy, halogen, nitro, amino, cyano, —C₁-C₆-alkyl, —C₁-C₄-hydroxyalkyl, —C₁-C₆-alkoxy, —C₁-C₄-alkyl-CO—NH—, —NH—C(O)—NH-aryl, —COOR⁵, —CR⁵(OH)—R⁶ and —CONH₂, and
m′, m″ are 0-3, independently of one another.

10. Quinoline derivative according to claim 9, wherein
R¹ and R² are the same or different and are selected in one or more places, independently of one another, from the group that comprises hydrogen, hydroxy, halogen, nitro, amino, cyano, —CH₃, —C₂H₅, CH₃O—, C₂H₅O—, HOCH₂—, CH₃CONH—, —NH—C(O)—NH-phenyl, —COOH and —CONH₂.

11. Use of the quinoline derivative according to claim 1 for the production of a pharmaceutical agent.

12. Use of the quinoline derivative according to claim 1 for the production of a pharmaceutical agent for treating diseases in which angiogenesis, lymphangiogenesis or vasculogenesis plays a role, for treating diseases of the blood vessels, for treating diseases that are caused by a hyperproliferation of body cells, as well as for treating chronic or acute neurodegenerative diseases.

13. Use of the quinoline derivative according to claim 1 for diagnostic purposes in vitro or in vivo for identifying receptors in tissues by means of autoradiography or PET.

14. Use of the quinoline derivative according to claim 1 as an inhibitor of the Eph-receptor kinases.

15. Use of the quinoline derivative according to claim 1 in the form of a pharmaceutical preparation for enteral, parenteral and oral administration.

16. Process for the production of the quinoline derivative according to claim 1 with the following process steps according to the diagram below: 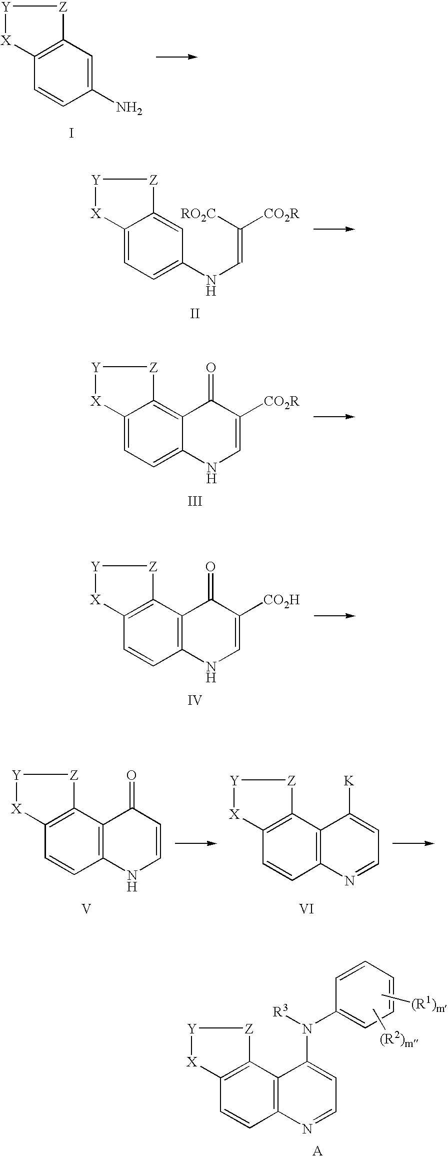
in which
K is selected from the group that comprises halogen and —OS(O)₂CₙF₂ₙ₊₁ with n=1-3,
R is methyl or ethyl, and
X, Y and Z have the same meaning as in general formula A
a) Addition of a compound with general formula I to a dialkyloxymethylene malonate with the formation of a compound with general formula II,
b) Cyclization of the compound with general formula II to the compound with general formula III,
c) Saponification of the compound with general formula III with the formation of a compound with general formula IV,
d) Decarboxylation of the compound with general formula IV with the formation of a compound with general formula V,
e) Reaction of the compound with general formula V with thionyl chloride or a perfluorosulfonic acid anhydride with the formation of a compound with general formula VI,
f) Addition of an amine with general formula (R¹)_{m′}, (R²)_{m″}ArNR³H, in which R¹, R², R³, m′ and m″ have the same meanings as in general formula A, to the compound with general formula VI with the formation of the quinoline derivative with general formula A.

17. Pharmaceutical agents that contain at least one quinoline derivative according to claim 1 as well as suitable formulation substance and vehichles.
